(19) United States
(12) Patent Application Publication
(10) Pub. No.: US 2013/0103139 A1
(43) Pub. Date: Apr. 25, 2013

(54) COATING OF ENDOPROSTHESES WITH A COATING CONSISTING OF A TIGHT MESH OF POLYMER FIBERS

(71) Applicants: Erika Hoffmann, Eschweiler (DE); Michael Hoffmann, Eschweiler (DE); Roland Horres, Stolberg (DE); Lutz Freitag, Hemer (DE)

(72) Inventors: Erika Hoffmann, Eschweiler (DE); Michael Hoffmann, Eschweiler (DE); Roland Horres, Stolberg (DE); Lutz Freitag, Hemer (DE)

(73) Assignee: HEMOTEQ AG, Wurselen (DE)

(21) Appl. No.: 13/702,174

(22) PCT Filed: May 27, 2011
(86) PCT No.: PCT/DE11/01152

§ 371 Date: Jan. 7, 2013

Related U.S. Application Data
(60) Provisional application No. 61/344,520, filed on Aug. 13, 2010.
(60) Provisional application No. 61/457,450, filed on Mar. 31, 2011.

(30) Foreign Application Priority Data
May 27, 2010 (DE) 102010022589.4
Jan. 21, 2011 (DE) 102011009053.3

(51) Int. Cl. A61F 2/82

(52) U.S. Cl. CPC A61F 2/82
USPC 623/1.46; 427/2.25

(57) ABSTRACT

The present invention relates to grid-like or net-like endoprosthesis having a continuous, respectively ongoing and interstices-spanning coating with a thread-tangle, wherein this continuous, respectively ongoing and interstices-spanning coating covers the struts as well as the interstices between the single endoprosthesis struts.

The present invention relates to endoprostheses coated with a polymeric close-meshed thread-tangle as well as the manufacture and use of the so coated endoprosthesis.

Pathological changes and injuries to the vascular walls in and at all body passages and body openings may lead to painful inflammations, constrictions, occlusions, sacculations and bleeding of these passage ways, so that the correct functioning of the hollow organ is impaired or even impossible. Degenerative diseases of the vascular walls represent with over 80% of the cases the most common cause for heart infarction or stroke in general. Poor nutrition, the widespread disease diabetes mellitus or also excessive smoking can lead to pathological and arteriosclerotic changes of the vascular passage, which can also manifest in the leg arteries and if not treated properly lead to necrosis and ultimately to amputation of the affected extremities.

Likewise life-threatening is the formation of aneurysms. These are sacculations of the vascular wall that can be traced back to an innate weakness of the connective tissue, arteriosclerosis, inflammations or traumas, or may be generated as the result of a volume load of the vascular wall. In this context it is mentionable that aneurysma spurium is also known as false aneurysm. Thereby a rupture goes through the intima and media of the vessel. This can be the result of a blunt or sharp injury, as it occurs after arterial puncture such as after puncture of the artery in the groin when conducting a PTCA and/or stent implantation as well as after heart catheter examinations. The probable reason therefore is assumably an insufficient pressure after removal of the catheter, so that the blood vessel is not closed properly leading to bloody oozing into the surrounding tissue.

Another and likewise commonly occurring danger affecting body passages is the growth of malignant and benign tumors. Rapid and uncontrolled cell division leads to the spreading of the tumor at and in hollow organs and thus to obstructions or occlusions of hollow body passages. Examples are esophageal cancer, cancer of the hypopharynx, nasopharynx and oropharynx, intestinal cancer, lung cancer, kidney cancer, occlusions of the bile duct, the pancreas and the urethra etc. Further causes for the impaired functioning of cavities can be cyst and fistula formation.

Stenosis in general refers to a physical obstruction or an interruption of the function of vascular cavities. Restenosis is a recurring stenosis, wherein the cause can be the initial treatment of a stenosis.

For treating constricted, blood-carrying body passages and for treatment of stenosis and restenosis, alongside the percutaneous transluminal angioplasty (PTA) or the percutaneous transluminal coronary angioplasty (PTCA), in the last two decades the stent has proven its worth as permanently in the body residing endoprosthesis with possibly locally acting active agent therapy. It is implanted directly with a balloon catheter and fixated during the PTA or PTCA, meaning during expansion of the affected site with a balloon catheter or after removal of the constriction at the affected site with atherectomy catheters. The stent, in its expanded form, presses the vascular wall outwards in a way that the native vessel diameter of the affected vessel is restituted and the vessel are kept open.

However, the foreign material of the endoprosthesis as well as the operation itself provokes protective reactions of the body. The endogenous defense system reacts thereupon within a short time through different paths such as humoral and specific immune reactions, hyperproliferation of cells, thrombus formation etc. that lead to an operation and therapy induced restenosis, if no further mitigating measures are taken.

Efforts in the continued development of endoprosthesis towards an improved biocompatibility of the used material, an increased flexibility combined with a reduced fatigue of material and a reduction of the foreign surface shall continuously minimize the risk of foreign surface-induced restenosis rate at least in the cardiovascular and peripheral vascular area.

Besides said basic requirements for such endoprosthesis with minimized foreign surface, the coating of the surface with biocompatible, biodegradable or biostable materials showed to be a promising advancement which mostly acts as a matrix for an anti-restenotic acting active agent. This active agent shall stop the pro-restenotic process by a time- and concentration-adjusted active agent release according to the requirements and ideally promotes the process of healing as good as in the ideal case of non foreign-influenced healing. Herein the requirements to the endoprosthesis itself, the coating material and the active agents as well as their interactions are equally high.

The same scaffold is used for relieving, preventing stenoses in all body passages, or for impeding the threatening obstruction as long as possible (such as in the palliative medicine or in the pain medicine), for example in the esophagus, bile duct, intestine, lung, kidney, urethra, pancreas, cerebral vessels, trachea (trachea bronchiale), paranasal sinus and other body cavities.

Hence, the task of the endoprosthesis is to stop the growth of excessive, malignant, benign and/or disturbing tissue in general into the lumen, preventing inflammations or reducing, preventing or remedying the risk of sacculation formation of hollow vessels. Alongside vascular restenosis caused by stents, furthermore, tumor growth, inflammations and aneurysm such as cyst formation, fistulas, traumas and scar formation shall be named as reasons for use of such endoprosthesis.

In contrast to vascular stents combating atherosclerosis these stents are hence provided with a preferably polymeric lining covering the entire cylindrical stent body including the interstices between the struts that should impede or at least delay also as an effective mechanical barrier the renewed ingrowth of the tumor through the interstices into the lumen.

It is common to all foreign materials used in body cavities that they ensure the highest possible unlimited flexibility, i.e. the physiologically necessary undisturbed, native motility of the target organ, and at the same time removing or delaying the occurred local disturbances of the hitherto normal conductivity. This flexibility is determined by the material and the design of the hollow body and has led to a wide-meshed, respectively net-like structure with a comparatively low vascular wall contact area.

According to symptoms and application site different requirements for the implant properties have to be taken into account. Thus for an endoprosthesis bound to be implanted into an artery there are different requirements than for example for an endoprosthesis destined to be implanted into the esophagus, bile duct, trachea, cerebral artery, paranasal sinus access, oropharynx, hypopharynx etc.

The vascular coated as well as the uncoated stent for the treatment of arteriosclerosis or stenoses and the prevention of stent-induced restenoses have the least possible foreign surface, as the currently commercially available products demonstrate.

There is a plethora of patent applications and patents in this field. As being effective, above all three competing stents are prevailing as market leaders. First, this is a polymer-coated stent eluting the active agent paclitaxel (Taxus stent from Boston Scientific Corp.), on the other hand a polymer-coated stent eluting the active agent rapamycin (Cypher stent from Cordis Corp.) as well as the stent Xience V (Abbott Vascular) eluting the sirolimus derivative everolimus.

Though the results and experiences with these and other coronary drug-eluting stents (DES) are very promising and represent a positive contribution to restenosis prophylaxis in the cardiovascular field not all problems are solved. For example, there is the phenomenon of in-stent restenosis such as late stent thrombosis (LST), as well as the finding of the optimal polymer. Despite good results the search for even more optimal active agents is going on in order to further reduce the restenosis rate as well as late complications.

An endoprosthesis used in tumor treatment can only constitute a barrier if it is able to cover the affected area completely, i.e. full-size coverage. This is only possible if the interstices of the surface minimized endoprosthesis don't remain passable, as only then the barrier is able to impede or retain tumor growth into the lumen.

As the polymer wrapped stent shall fulfill its function adapted to the site of action in a safe manner and in the ideal case shall ensure or at least support, but not bias in a negative way or even disturb the unhampered function of the target organ, different concepts have been elaborated in the past through which a stent shall be provided with a polymeric sleeve.

Thus WO 93/22986 describes a self-expanding esophagus stent which is covered with a silicone tube on its central section and which compresses this section in such a way that the stent has a lesser diameter than the tube-free proximal and distal end sections. The proximal and distal ends are not covered for enabling a better fixation of the stent to the cavity walls by means of the free stent struts. But this stent didn't turn out to be successful because problems are arising by the constriction of the stent body, for example during vomiting the forces acting on the stent are so increased that the stent is moved and injures the esophageal wall with its free stent ends.

Further the silicone tube can be torn or it can detach under these circumstances and mucus or food particles can settle between the vascular wall and the silicone coating so that apart from the threat of inflammation several scenarios utterly negative for the patient may become realistic.

WO 2005/030086 describes a method for full-size coating of a likewise self-expanding stent body with a polyurethane sleeve in which after a first spray coating of the stent with a polymer the polymer is imposed to the struts from the inside as a foil by means of a balloon or another suitable cavernous template. Herein the coating covering the entire stent occurs from the luminal side so that on the exterior side the stent struts keep on stabilizing the stent in the wall of the cavity. The subsequent heating of the system beyond the softening temperature shall bind the polyurethane to the stent. Problems arise since the polymeric sleeve is not quantitatively or completely bound to the coated stent and therefore does not remain permanently on the stent under the given circumstances. Likewise small holes may form through the heating that in the case of implantation may possibly enlarge and finally may lead to a detachment of the coating material and even to a delocalization of the entire stent.

Furthermore, the heating beyond the softening temperature of the polymer may lead to a situation wherein on the one hand the coating on the abluminal surface of the stent struts softens and invades the interstices between the struts and thereby the polymer coat does not only adhere to the stent, but also to the balloon likewise consisting of a polymer, so that during dilatation the coating can rupture or the stent does not detach from the balloon. Thus on retracting the balloon the interior coating has adhesion problems and is detached at least partially when the balloon is removed from the stent. As a result, food or mucus can settle between the detaching coating and the interior wall that step-by-step severs the coating from the stent but above all hampers the undisturbed passage. The detaching material stands out into the cavity and leads to additional irritations, nausea or cough which supports or rather is the cause for defixation of the entire stent. Currently, a commercially available esophagus stent is the ALIMAXX-ES™, which is a completely encased vascular support with a smooth PU-polymer sleeve (as foil).

A further field of application of stent-strut-interstices-overlapping coated stents is in the field of tracheal stenoses, most commonly caused by bronchial carcinomas, which are currently holding the second place in industrialized states in the ranking of the incidence rate of malignant tumors. These tumors can hardly be healed by surgery or by means of a multimodal therapy so that ca. 30% of the patients diseased of a stenosis of the central airways also die on it.

A special problem in this field arises from the shape of the trachea which is not round, in contrast to other hollow passages, so that the risk that a stent detaches itself and likewise that mucosa gathers between the coated stent and the tracheal wall is particularly high for these stents. A similar unfavorable situation results when the coating detaches from the stent under the given circumstances and secretion may settle between stent and coating. The risk of detachment of the coating has to be taken into account for all coated vascular supports to the same degree and in all fields of application, also cardiovascular.

Most commonly, the so-called Dumont stent is still used, a tubular silicone tube with naps for a better fixation on the abluminal side, specifically developed for the trachea area, since it can be removed more easily in contrast to most metal stents, because due to commonly occurring subsequent complications re-implantation is often necessary.

The different commercially available metal stents (e.g. the nitinol stent, gianturco and wall stent) are nowadays often used in a full-size coated form but likewise still don't show the desired success.

Because of the conditions in the trachea the migration of a foreign body is still an improvable problem. In addition to the poor fixation comes a disadvantageously high wall thickness, as present e.g. in the Dumont stent, impeding the secretion flow along the interior wall surface, i.e. luminal. This causes an accumulation of secretion by which the air stream is impeded again, which leads to inflammations and favors colonization by germs.

These “restenoses” are a commonly occurring complication. Thus there is a stent-induced restenosis risk not only with the conventional drug-eluting stent (DES) in the coronary field but also for full-size coated products, i.e. consistently coated products such as a tube, this substantial risk of a new occlusion or constriction of the coated stent with e.g. bronchial secretion has to be taken into account which in the end can only be removed surgically as a viscous rubber-like mass.

Another common cause for the occlusion or the increased adhesion of mucosa lies in the desiccation of the luminal stent surface since the body-regulated moisture of a native interior wall is not given anymore but is necessary for allowing the bronchial secretion to flow off. It adheres in this dry area and thus is accumulating ever the more, as the breathed air alone can't maintain the necessary moisture in this segment for ensuring a natural equilibrium, as is warranted by the mucous membranes. Thus the affected patients depend upon the regular inhalation of liquid nebulizers in order to delay the infallibly occurring obstruction with secretion as long as possible.

Another and for the patients utterly unpleasant social problem is the extremely malodorous breath caused by the in situ colonization of bacterial germs on the implant surface, since the colonization by germs at these sites can't be averted anymore under the given circumstances. Locally occurring inflammations of the most diverse origin but also as a result of the stent implantation are likewise causal for a new occlusion.

The AERO® stent from Alveolus tries to contain this problem, but is no yet fully developed. The stent also has a very smooth foil-like coating material such as the esophagus stent ALIMAXX-ES™ already described above.

The same scaffold of a stent coated with some kind of foil can be used for treatment of aneurysm. The cause of aneurisms is the pathologic sacculation of the vascular wall in which blood is gathering and coagulating. Due to the weight load the vascular wall stretches ever more at this site, resulting in further blood flow, stagnation and clotting. Besides the increasing threat of thrombosis this finally leads to a vascular rupture.

U.S. Pat. No. 5,951,599 envisages to solve this problem by filling the free interstices of a vascular stent with a small-meshed partially applied polymeric network which is positioned over the sacculation in the blood vessel and will cover the aneurysm in such a way that the blood flow comes to a standstill in the sacculation. As a consequence a stable thrombus is formed therein, thereby stopping the enlargement of the aneurysm. Further, the polymeric coverage shall prevent that the thrombus or parts of the clot are spilled into the blood circulation and can cause an infarction elsewhere. Here the same problems arise, too, because of bad adhesion of the polymeric network which deprives the stent of its function and thus leads to an increased risk for the patient. Currently, aneurysms are still treated by filling them with metal wire (“coils”) which shall stop the blood flow inside the sacculation. But also the commonly and necessarily used artificial inlets or artificial outlets to hollow body organs are insufficient, when used for longer time periods of time. Painful inflammations and bacterial infections result in frequent changes of the inlets and thereby to complications and additional intolerable and risky stress for the patient. Hence, it is important to find a solution that assures safety of the patient.

It is the objective of the present invention to provide a coated endoprosthesis and in the case of endoprosthesis with interstices such as stents to provide interstices-overlapping or interstices-covering coated endoprosthesis, which avoid the described disadvantages for all body passages including the coronary fields of application and which under consideration of the conditions existing at the application site provide an optimal, uniform production method for such implants.

This task is solved by the technical teaching of the independent claims of the present invention. Further advantageous embodiments of the invention result from the dependent claims, the description and the examples.

It was found that the problems of the state-of-the-art can be solved by means of an endoprosthesis the surface of which has a coating of a thread-tangle. The coating is preferably a sprayed thread-tangle. Hence, an inventive endoprosthesis has a surface coated at least partially or completely with a polymeric close-meshed or tight-meshed thread-tangle. Moreover it is preferred, if the thread-tangle coating, i.e. the coating of thread tangle, reaches over the ends of the endoprosthesis and thereby covers sharp edges or prevents exposed strut regions.

The thread-tangle coating is flexible, mechanically stable and consists of a polymeric material consisting of threads, which are oriented statistically and randomly and are tangled and linked with each other and have meshes that are formed by the surrounding threads. The single threads of the thread-tangle coating consist of the polymeric material and in particular of the herein mentioned polymers. These polymers have preferably a high average polymerization grade.

This thread-tangle can be applied as coating to full-size, tubular endoprosthesis such as bladder catheters, bypasses and artificial stomae outlets as well as on so called stents. A stent is to be understood as a grid-like or net-like endoprosthesis. A stent does not form a massive tube but a grid-network. A stent for example is cut out of a massive tube e.g. by means of a laser, leaving only single preferably thin struts connected together. The term “struts” as used herein shall be understood as single solid segments (stent struts) of the scaffold of the endoprosthesis or stent that are interconnected at nodes and thereby form the expandable and flexible structure of the endoprosthesis.

On cutting a stent segments between the single struts are cut out which shall be named “interstices” herein. Thus an endoprosthesis has a plurality of solid scaffold components (e.g. struts, in form of rings, spirals, waves and wires) that build the endoprosthesis, as well as a plurality of interstices between the solid components. In common embodiments of endoprostheses the struts converge in nodes so that the interstices are defined by the surrounding struts and nodes. There are, however, endoprosthesis embodiments having no or nearly no nodes and the struts having for example the form of rings or spirals. In such endoprostheses there is for example partially no plurality of interstices anymore but only a few or only one interstice defined for example by two intertwining spirals. Then such interstices are not completely bounded anymore but can have one or two or also more open ends or open sides. Anyway, “interstices” refer to the open or bounded area between the solid endoprosthesis components.

A thread-tangle coating according to the invention is applied interstices-overlapping on a stent, i.e. the interstices formed by the interstices enclosing struts are also coated. Thus, this coating spans the interstices of the single struts, like a bridge, which is only tethered at the scaffold, the struts, and the interstices do not rest on solid ground. A thus generated lining may refer to the entire cylindrical stent body or only as to selected areas thereof. For example, optionally either proximal or distal segments, the central section, single segments or stents coated half-side in longitudinal direction and of course also combinations of these areas can be coated, according to the indication. The coating is applied preferably on the outer side, i.e. the side facing away from the lumen (abluminal). But depending on the indication the lumen facing side can also be coated with a coating of a polymeric close-meshed or tight-meshed thread-tangle. It is also possible to coat both sides.

The term “interstices-overlapping” as used herein refers also to interstices-spanning or interstices-covering and hence clarifies that in comparison to other coated stents the coating is not only around the stent struts, but is all around the whole stent. This can be seen especially well in FIG. 3 and FIG. 7C. FIG. 3 shows a thread-tangle coating around a stent and the luminal metallic surface of the stent struts can be seen through the torn open parts. Furthermore, it can be seen that the coating of the thread-tangle is not around the single stent struts but only adjoining at the abluminal surface of the stent struts wrapped around the whole stent. FIG. 7C shows how the coating of thread-tangle covers the whole stent like a textile coat and the stent pattern pressing lightly from inside of the thread-tangle coating is well recognizable.

For the coating are used supports for all body passages or body cavities, commonly also named “vessels”, such as arteries, veins, esophagus, bile ducts, kidney ducts, hollow passages in the nose and mouth region, trachea, bronchial channels, duodenum segments, colon or other approximately tubular body passages, wherein this preferable group of endoprosthesis has a grid-like or net-like structure, as for example a stent. The term “body passages” or “vessels” comprises herein not only natural body passages or body channels but also artificial body openings and body channels as for example bypasses and artificial stomae. Further applications for endoprosthesis coated according to the invention thus are larynx implants, bypasses, catheters or artificial stomae and in general all areas in or at the living organism where the body passage has to be kept free as well as motile, wherein the vascular walls are not isolated completely from the lumen side, so that the necessary contact between the inner vessel wall and the lumen is ensured. By this way an isolation of the cavity wall from the lumen is prevented concerning the important substances in the lumen that are necessary for the preservation of the health of the inner cavity surface. The permeable coating allows the exchange, transport and delivery of substances that are important for the preservation of function between lumen and cavity surface such as liquids, moisture, nutrients or molecular substances necessary for preservation of the function. Thereby the impact of the implanted foreign body on the surrounding is reduced to a minimum.

Such a coated endoprosthesis can be adapted for individual applications by thread diameter, thread length, mesh number and mesh size, pore size and pore formation, degree of cross-linking and inter- and eventually intrafilamentary permeability of the tangle according to corresponding needs in the target vessels.

A thread-tangle as well as a thread-tangle coating consists of loosely and randomly arranged fibers or threads that because of their confuse and random unorganized structure are difficult to be separated into single fibers or threads. The consistency of a thread-tangle and of the thread-tangle coating thus depends on the adhesion intrinsic to the fibers and on the confuse, random and unorganized structure. The thread-tangle can be additionally solidified to which end different methods can be used such as temperature, light, moisture and/or pressure. A solidified thread-tangle is preferred as coating in the organism because detachment of threads that could cause complications is prevented thereby. The mutual adhesion of the threads and thus the solidification results herein in the ideal case already during the drying procedure through the evaporation of the solvent. Also after the drying procedure the thread-tangle coating is tearproof, expandable and compressible, respectively crimpable (i.e. able to be mounted on a catheter balloon). Sterilization of the endoprosthesis (heat sterilization with hot air and steam, fractionized sterilization or chemical sterilization with ETO, ozone, formaldehyde, hydrogen peroxide or peracetic acid) must also be possible without having any influence on the structure or permeability of the thread-tangle however the method must be adapted to the properties of the used material of the endoprosthesis.

A thread-tangle according to the invention is a textile planar product of single fibers or threads that are not interweaved, knitted or braided or are otherwise connected or jointed in a specific pattern with each other. In contrast, tissues, knitted and weaved fabrics, are produced of yarns and membranes of foils, underlying certain order principles and knitting mechanisms.

In contrast, fibrous coatings of the thread-tangles consist of fibers or threads the position of which can only be described with statistic methods. The threads also referred to as fibers are arranged in a confuse, disorderly and random manner to each other. The openings that arise between the threads are designated as meshes.

The term “mesh” as used herein describes an opening between the surrounding threads of the thread-tangle coating. The openings are not necessarily round but can assume any shape because the threads of the thread-tangle coating are oriented and spread in a random manner. So an opening, i.e. a mesh is usually surrounded by several threads. Moreover, the meshes show a certain size distribution. The longitudinal diameter of a mesh is to be understood as the maximum extension of this opening and the transverse diameter is the minimal extension of this opening. The cross-sectional area of a mesh is to be understood as the area of this opening, i.e. of this mesh within the surrounding threads. Furthermore, the entireties of the meshes also have an average longitudinal diameter as well as an average transverse diameter as well as an average cross-sectional area. These are the averaged values of the above defined factors over the entirety of the meshes. The determination of the number, area and diameter of the single meshes can be done by spectroscopic methods.

In FIG. 4 a wedge-shaped mesh can be seen centrally arranged (dark area in the middle of FIG. 4, the tip of the wedge pointing to the right), which is smaller than the tumor cell lying underneath (the brighter area beginning directly under the mesh, extending downwards oval and long-stretched) so that the tumor cell cannot pass the thread-tangle coating.

The threads of the thread-tangle coating have an average thread diameter in the range of 1 μm to 30 μm, preferably in the range of 1 μm to 20 μm, further preferred in the range of 1 μm to 15 μm, even more preferred in the range of 1 μm to 10 μm and in particular preferred in the range of 2 μm to 7 μm.

The meshes of the thread-tangle coating have an average diameter in the range of 0.01 μm to 1000 μm, preferably in the range of 1 μm to 1000 μm, further preferred in the range of 10 μm to 500 μm, even more preferred in the range of 25 μm to 250 μm and in particular preferred in the range of 50 μm to 150 μm.

The meshes of the thread-tangle coating have a certain size distribution, wherein size is referred to as the cross-sectional area of each single mesh in a vertical top view on the respective mesh and the thereby obtained two-dimensional display.

According to the invention the endoprosthesis can be coated with a thread-tangle consisting of a preferably linear polymer or a mixture of polymers that may be biodegradable or biostable. The polymer(s) can be selected from the group comprising or consisting of:

Polyurethane, polyethylene terephthalate, polyvinyl chloride, polyvinyl ester, polyvinyl acetals, polyamides, polyimides, polyacryl-nitriles, polyethers, polyesters such as poly-3-hydroxy butylates, poly-3-hydroxy alkanoates, polyamino acids, polysaccharides, polylactides, polyglycolides, polylactide glycolides, chitosans, carboxyalkyl chitosans such as carboxymethyl chitosans, collagen, polyphosphazenes, polystyrenes, polysulfones, silicones as well as derivatives, block polymers, co-polymers and mixtures of the afore-mentioned polymers. In principle, all polymers that are biocompatible, not cross-linked and soluble in a solvent can be used.

The present invention thus relates to methods of coating of biostable or biodegradable endoprostheses, in particular stents, but also of other prosthesis and auxiliary materials that remain for longer periods in the body, wherein these are coated with a polymeric close-meshed or tight-meshed thread-tangle.

Thus the invention also comprises methods for the coating of an endoprosthesis for expanding a vascular lumen, comprising the following steps:
a) providing an endoprosthesis,
b) solving a polymer in a volatile solvent,
c) applying a polymer-based thread-tangle by means of spraying or electro spinning on the surface of the endoprosthesis.

Besides spray coating the coating can be also carried out by means of electro spinning, wet spinning or melt spinning.

As solvents, preferably those solvents are used that solve the polymer well and are volatile. As solvents, solvents with a high vapor pressure are preferably used, such as acetone, butanone, pentanone, tetrahydrofuran (THF), benzene, toluene, light petrolether, dimethyl formamide (DMF), dimethyl sulfoxide (DMSO), xylene, ethylene glycol, water, methanol, ethanol, propanol, chloroform, methylene chloride, acetic acid ethyl ester, n-hexane, isopropanol, phenol or their mixtures.

In this inventive method the clogging of the threads of the thread-tangle occurs by the threads themselves, meaning the threads generated only by spraying the solution, with still adhesively moist surface adhere upon contact against and above each other and herein also additives such as active substances can be incorporated into the thread-tangle, which aren't adhesive or at least don't have to be adhesive. Thus no additional adhesive, cross-linking steps or cross-linking agents are needed that would considerably modify the thread surfaces. The threads of the thread-tangle rather clog due to the presence of the still sticky threads from the solvent at their contact points resulting in a thread-tangle according to the invention. Thus no dissimilar adhesive is needed that would cover the fiber surfaces so that the fiber-specific effects wouldn't develop. By self-implementing the cohesion of the thread-tangle by fibers only clogged at the crossing points the thread-tangle structure displays also better capillary characteristics that favor the absorption of fluid and moisture. Spraying the solution for thread generation can preferably be carried out by compressed air nozzles. The structure of the thread-tangle and the thread diameter can be varied by material pressure, variations in nozzle outlets, distance between endoprosthesis and nozzle as well as by polymer concentration. Since the threads are only clogged at their contact points the whole thread-tangle coating is more flexible and mobile, whereby rupture of the thread-tangle coating during dilatation is avoided.

The thread-tangle coating can be preferably extended up to 10% of its length without the occurrence of flaws, further preferred up to 100% of its length, further preferred up to 200% and in particular preferred extended up to 400% of its length, without the occurrence of flaws.

The thread-tangle coating of the inventive endoprosthesis preferably has a porosity defined as air permeability of 1 to 150 ml [1 to 150 ml/(cm²*60 s)], more preferred of 10 to 100 ml [10 to 100 ml/(cm²*60 s)] and particularly preferred as 20 to 50 ml air per square centimeter per minute [ml/(cm²*60 s)] at a pressure difference of 1.2 kPa.

The thread-tangle coating of the inventive endoprosthesis preferably has a porosity defined as water permeability in the range of 100 to 300 ml/cm²*min, particularly of 150 to 250 ml/cm²*min (ml water per square centimeter and per minute at Δp=120 mmHg). These water permeability values were measured according to Weselowski's method of determination at 120 mm Hg. An inventive endoprosthesis is preferably characterized by the inventive thread-tangle having meshes and consisting of porous threads.

These features can be used and adjusted upon requirement so that essential modalities and multiple possibilities result for the used polymeric materials and the resulting coated endoprosthesis. Besides the used polymer or polymeric mixture, key parameters are the thread diameter, the thread porosity, a varying coating thickness, the mesh cross-section, the spraying technique, the solvent etc. Despite of the same coating procedure these numerous variation options ensure an endoprosthesis that is optimally and individually applicable in all known vascular diseases.

For example, the thread-tangle coating can be realized in such a way that a tumor cell has no possibility to intrude between the threads into the inner lumen (see FIG. 4). Furthermore, this coating mode prevents that e.g. the luminal surface of the hollow organ e.g. can dry out since the adjustable size of the meshes promote a further provision of the interior surface with moisture, because the thread-tangle coating does not separate the interior surface of the hollow organ or body passage like a continuous impermeable foil from the interior lumen of the endoprosthesis, but only excludes the passaging of bigger particles or of cancer cells, but not the permeation of liquid, water or air. Stents with a polymeric film-like full-size coating show exactly these disadvantages, because exchange of moisture or air is prevented. Whereas the coated stents according to the invention allow the necessary exchange processes between vascular wall and lumen and ensure that the stented interior vascular wall area is not isolated of necessary processes and/or substances and thus the healing process is supported optimally. According to the field of application the germ-killing processes of the own body can prevent or reduce the problematic of germ development.

According to the field of application a further coating on the luminal side of the inventive coated stent with hydrophilic polymers may be supportive.

Likewise a smooth luminal surface can also be desirable such as with a trachea stent, so that the flow of the mucosa is ensured. This can be easily achieved during application of the inventive coating on the endoprosthesis by mounting the endoprosthesis on a cylindrical metal core adapted to the diameter of the endoprosthesis, so that no threads can protrude into the lumen but nevertheless the thread-tangle structure is formed perfectly luminal as well as abluminal. For easier detachment of the spray coated endoprosthesis from the metal core, eventually wetting of the interior side with a solvent might be necessary or stents with lubricated pre-coated stent struts are used for coating.

In further preferred embodiments thread-tangles are used or applied according to the invention which further contain at least one antiproliferative, antimigratory, anti-angiogenic, anti-inflammatory, anti-restenotic, antiphlogistic, cytostatic, cytotoxic and/or anti-thrombotic agent. This active agent can be contained in a covalently bounded form, or in an adhesively or ionically bounded form. Thereby coated medical products, respectively endoprosthesis are obtained that contain at least one active agent in the thread-tangle coating, preferably in the form of a drug-releasing coating (drug release system). The thread-tangle coating can be manufactured by dissolving the active agent or the active agent mixture in the spraying solution and then applying the spraying solution or alternatively by applying it afterwards to the thread-tangle coating.

It is advantageous herein that the release of the active agent or the active agent mixture out of the inventive thread-tangle coating does not only occur were the stent struts are, which is the case with common stents, but also over the entire diseased area, where the inventive coated endoprosthesis is implanted. In contrast to commercially available current drug-eluting stents that are only coated with active agent in the area of the struts, this leads to a comprehensive provision of the diseased site with the necessary remedies and not only to a punctual treatment of the affected sites, or even to the treatment of areas close to a lesion only. Likewise, in comparison to the even coating of the struts of conventional stents the rather raw thread-tangle texture is helpful for the colonization of the injured areas with new cells, as their adhesion is facilitated.

The following advantages can be listed for endoprosthesis coated with an inventive coating of a polymeric close-meshed or tight-meshed thread-tangle:
1. The coating method is universally applicable for the area of vascular endoprosthesis as well as artificial body passages such as artificial stomae outlet, bladder catheter, vein catheter, in short all artificial in- and outlets at or in the body necessary for a longer period of time and still individually adjustable to different conditions by the choice of the polymer material, addition of active agents and the adjustable process parameter such as mesh size or pore size of the threads.
2. The thread-tangle covers the generated non-evenness of the body passages in the lesioned area and thus provides a significant and necessary protection e.g. in the case of a vascular stent from thrombocyte attachments in the lesioned area and thus constitutes a significant inhibition of the coagulation cascade initiated by activated thrombocytes, with a resulting life-threatening hemostasis.
3. The lesioned area of the vascular wall is substantially protected by the thread-tangle coating from activities inside the cavity so that the healing processes can occur in an optimal manner.
4. The polymeric close-meshed or tight-meshed thread-tangle coating provides for an additional stability of the body passages in the lesion area.
5. The polymeric close-meshed or tight-meshed thread-tangle coating serves as a mechanical barrier against hyperproliferation, tumor growth, new fistula formation and formation of cysts as well as external bleedings.
6. Via the still permeable thread-tangle structure the at least minimal contact between lumen and vascular wall is maintained, so that the most necessary requirements such as permeation of nutrients, moisture, oxygen etc are possible, albeit to a limited extent.
7. The textured surface of the thread-tangle coating provides for an additional support of the endoprosthesis in the vascular wall.
8. The polymeric close-meshed or tight-meshed thread-tangle coating provides for a reasonable even distribution of the added active agent over the entire affected area.
9. The significantly larger surface of the thread-tangle coating of a polymeric close-meshed thread-tangle allows the application of an increased amount of the active agent.
10. Through the significantly larger surface of the coating of a polymeric close-meshed thread-tangle also those active agents can be administered that only lead to a successful treatment over a certain dosis that couldn't be realized with a coating of the struts only. Thus the inventive coating can broaden the choice of suitable active agents in a most simple manner.
11. Active agents can be mixed directly into the spraying solution for the thread-tangle forming polymers.
12. Active agents can be introduced afterwards by filling the meshes formed by the threads of the thread-tangle.
13. The active agents elute with different speed.
14. Active agents can be separated locally from each other, on one side in the porous or biodegradable polymeric fibers and on the other side between the thread-tangle forming threads.
15. The distribution of the active agents over the entire endoprosthesis is absolutely uniform despite of local separations.
16. Different active agents can be introduced locally separated, which both are still uniformly distributed and eluted over the whole therapeutic area.
17. The luminal side of such a coated endoprosthesis can be smooth, coated or uncoated, with or without active agent, according to the needs.
18. The coating of a polymeric close-meshed or tight-meshed thread-tangle as well as the texture offer a significantly larger surface for the most diverse approaches for the treatment of a lesion in the vascular walls of a body passage than a common (only on the struts) coated endoprosthesis.
19. The partial application of the polymeric close-meshed or tight-meshed thread-tangle coating allows for a specific treatment of the diseased site, e.g. a tumor growing into the lumen from the right side can be stopped with a stent that was coated only on this side. The opposite side of the endoprosthesis stays open or will be coated only on the struts. This variant is also well suitable for treatment of aneurysm.
20. The pores formed by the thread-tangle can not only be filled with active agents but if the need arises can be filled with other materials and excipients that elute after a short time together with the active agent or are degraded. Rapidly degrading polymers as well as active agent carriers and elution controls can be used just as active agent transfer-accelerators so called transport mediators or mediators.

Finally, in case of sufficient, preferably time-limited stability the polymeric close-meshed or tight-meshed thread-tangle coating can also be used even without an endoprosthesis. For this purpose the optionally active agent containing thread-tangle is sprayed directly on a moulding core. Besides a stent, also ongoing, full-size and tubular endoprosthesis can be coated. Therefore, the thread-tangle optionally containing an active agent is directly applied to the endoprosthesis (for example in case of a bladder catheter) or the transport unit. Endoprosthesis remaining temporary in the organism, such as bladder catheters or vein catheters coated with the thread-tangle and e.g. equipped with antibacterial or anti-inflammatory active agents could solve or at least significantly improve the problems of many patients with permanent catheters.

Such a thread-tangle as coating of a degradable or biodegradable endoprosthesis could slowly degrade under controlled conditions after a preset time without any long-term complications that for example are accompanied with non-degradable endoprosthesis.

Likewise useful is a biostable or biodegradable thread-tangle on a biodegradable stent. Depending on the field of application a biodegradable thread-tangle can also be advantageous on a removable implant e.g. the removal of the endoprosthesis after degradation of the biodegradable thread-tangle. The coating and endoprosthesis can also be configured to be biodegradable. Also in this case the use of an active agent could be reasonable.

Naturally it must be ensured that the coating of a polymeric close-meshed or tight-meshed thread-tangle does not release any fragments or particles that could lead to life-threatening situations.

Of course, it is also possible to apply the active agent(s) in a separate coating step either directly on the surface of the endoprosthesis and thereby under the thread-tangle coating or on the thread-tangle coating or under as well as on the thread-tangle coating.

The active agent concentration is preferably in the range of 0.001-500 mg per square centimeter coated endoprosthesis surface, i.e. the surface is calculated in account of the total surface of the inventive thread-tangle coating.

According to the coating method, the active agent(s) can be situated under, in and/or on the thread-tangle coating. As antiproliferative, anti-inflammatory, antimigratory, antiphlogistic, anti-angiogenic, cytostatic, cytotoxic, anti-restenotic, anti-neoplastic, anti-bacterial and/or anti-mycotic agent can be used preferably:

Abciximab, acemetacin, acetylvismione B, aclarubicin, ademetionine, adriamycin, aescin, afromoson, akagerine, aldesleukin, amidorone, aminoglutethemide, amsacrine, anakinra, anastrozole, anemonin, aminopterine, antimycotics, antithrombotics, apocymarin, argatroban, aristolactam-All, aristolochic acid, ascomycin, asparaginase, aspirin, atorvastatin, auranofin, azathioprine, azithromycin, baccatine, bafilomycin, basiliximab, bendamustine, benzocaine, berberine, betulin, betulinic acid, bilobol, bisparthenolidine, bleomycin, bombrestatin, Boswellic acids and derivatives thereof, bruceanoles A, B and C, bryophyllin A, busulfan, antithrombin, bivalirudin, cadherins, camptothecin, capecitabine, o-carbamoyl-phenoxy-acetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type Natriuretic Peptide (CNP), cudraisoflavone A, curcumin, cyclophosphamide, cyclosporine A, cytarabine, dacarbazine, daclizumab, dactinomycin, dapson, daunorubicin, diclofenac, 1,11-dimethoxycanthin-6-one, docetaxel, doxorubicin, daunomycin, epirubicin, epothilones A and B, erythromycin, estramustine, etoposide, everolimus, filgrastim, fluoroblastin, fluvastatin, fludarabine, fludarabine-5′-dihydrogenphosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mitoxantrone, mycophenolate mofetil, β-lapachone, podophyllotoxin, podophyllic acid 2-ethylhydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies which inhibit muscle cell proliferation, bFGF antagonists, probucol, prostaglandins, 1-hydroxy-11-methoxycanthin-6-one, scopoletin, NO donors, pentaerythritol tetranitrate and sydnonimines, S-nitrosoderivatives, tamoxifen, staurosporine, β-estradiol, α-estradiol estriol, estrone, ethinylestradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and its derivatives, 6-α-hydroxy-paclitaxel, taxotere, mofebutazone, lonazolac, lidocaine, ketoprofen, mefenamic acid, piroxicam, meloxicam, penicillamine, hydroxychloroquine, sodium aurothiomalate, oxaceprol, 6-sitosterin, myrtecaine, polidocanol, nonivamide, levomenthol, ellipticine, D-24851 (Calbiochem), colcemid, cytochalasin A-E, indanocine, nocadazole, bacitracin, vitronectin receptor antagonists, azelastine, guanidyl cyclase stimulator tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1, active agents from the group of antibiotics, cefadroxil, cefazolin, cefaclor, cefoxitin, tobramycin, gentamycin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyridamol, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, paracetamol, dexpanthenol, clopidogrel, acetylsalicylic acid derivatives, streptomycin, neomycin, framycetin, paromomycin, ribostamycin, kanamycin, amikacin, arbekacin, bekanamycin, dibekacin, spectinomycin, hygromycin b, paromomycinsulfate, netilmicin, sisomicin, isepamicin, verdamicin, astromicin, apramycin, geneticin, amoxicillin, ampicillin, bacampicillin, pivmecillinam, flucloxacillin, mezlocillin, piperacillin, azlocillin, temocillin, ticarcillin, amoxicillin, clavulanic acid, ampicillin, sulbactam, piperacillin, tazobactam, sulbactam, cefamandol, cefotiam, cefuroxim, cefmenoxim, cefodizim, cefoperazon, cefotaxim, ceftazidim, cefsulodin, ceftriaxon, cefepim, cefpirom, cefoxitin, cefotetan, cefalexin, cefuroxim axetil, cefixim, cefpodoxim, ceftibuten, imipenem, meropenem, ertapenem, doripenem, aztreonam, spiramycin, azithromycin, telithromycin, quinopristin, dalfopristin, clindamycin, tetracycline, doxycyclin, minocyclin, trimethoprim, sulfamethoxazol, sulfametrol, nitrofurantoin, lomefloxacin, norfloxacin, ciprofloxacin, ofloxacin, fleroxacin, levofloxacin, sparfloxacin, moxifloxacin, vancomycin, teicoplanin, linezolid, daptomycin, rifampicin, fusidic acid, fosfomycin, trometamol, chloramphenicol, metronidazol, colistin, mupirocin, bacitracin, neomycin, fluconazol, itraconazol, voriconazol, posaconazol, amphotericin b, 5-flucytosin, caspofungin, anidulafungin, tocopherol, tranilast, molsidomine, tea polyphenols, epicatechin gallate, epigallocatechin gallate, leflunomide, etanercept, sulfasalazine, etoposide, dicloxacillin, tetracycline, triamcinolone, mutamycin, procainimide, retinoic acid, quinidine, disopyramide, flecamide, propafenone, sotolol, natural and synthetically obtained steroids, inotodiol, maquiroside A, ghalakinoside, mansonine, strebloside, hydrocortisone, betamethasone, dexamethasone, non-steroidal substances (NSAIDS), fenoprofen, ibuprofen, indomethacin, naproxen, phenylbutazone, antiviral agents, acyclovir, ganciclovir, zidovudin, clotrimazole, flucytosine, griseofulvin, ketoconazole, miconazole, nystatin, terbinafine, antiprotozoal agents, chloroquine, mefloquine, quinine, natural terpenoids, hippocaesculin, Barringtogenol-C21-angelat, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, Coronarin A, B, C und D, ursolic acid, hyptatic acid A, iso-iridogermanal, maytenfoliol, effusantin A, excisanin A and B, longikaurin B, sculponeatin C, kamebaunin, leukamenin A and B, 13,18-Dehydro-6-alpha-senecioyloxychaparrin, taxamairin A and B, regenilol, triptolide, cymarin, hydroxyanopterin, protoanemonin, cheliburin chloride, sinococuline A and B, dihydronitidine, nitidine chloride, 12-beta-hydroxypregnadien-3,20-dion, helenalin, indicine, indicine-N-oxide, lasiocarpine, inotodiol, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, bisparthenolidine, oxoushinsunine, periplocoside A, ursolic acid, deoxypsorospermin, psycorubin, ricin A, sanguinarine, manwu wheat acid, methylsorbifolin, sphatheliachromen, stizophyllin, mansonine, strebloside, dihydrousambaraensine, hydroxyusambarine, strychnopentamine, strychnophylline, usambarine, usambarensine, liriodenine, oxoushinsunine, daphnoretin, lariciresinol, methoxylariciresinol, syringaresinol, sirolimus (rapamycin) and its derivatives such as biolimus A9, everolimus, myolimus, novolimus, pimecrolimus, ridaforolimus, deoxorapamycin, tacrolimus FK 506, temsirolimus and zotarolimus, somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin and sulfur containing amino acids such as cystine as well as salts, hydrates, solvates, enantiomers, racemates, enantiomer mixtures, diastereomers mixtures; metabolites, prodrugs and mixtures of the aforementioned active agents.

The thread-tangle coating or the meshes of the thread-tangle coating may be sealed with a resorbable or under the working conditions resistable impregnation. These can also contain an active agent, which is released in a controlled manner. Furthermore, the meshes formed by the thread-tangle can be filled with a resorbable polymer or oligomer or a viscous substance, containing an active substance or being itself the active substance.

Furthermore, in a step anterior to the coating step with the thread-tangle a hemocompatible layer can be immobilized on the surface preferably bound in a covalent manner on the uncoated endoprosthesis surface, or by cross-linkage e.g. with glutaraldehyde. Such a layer that doesn't activate blood coagulation is reasonable in those cases when uncoated stent material may come into contact with blood. Thus it is preferred to provide a partially coated stent with this interior hemocompatible layer first. Alternatively, also an exterior, optionally hemocompatible layer can be applied on the thread-tangle coating. “Interior” layer or coating indicates the layer or coating which is applied directly on the stent surface. “Exterior” layer or coating indicates the layer or coating which is the top one or the most distant one from the stent surface.

The preferably hemocompatible layer is produced from the following preferred materials: Heparin of native origin as well as regioselectively produced derivatives of different degrees of sulfatation and acetylation in the molecular weight range of the pentasaccharide responsible for the antithrombotic effect to the standard molecular weight of commercially available heparin of ca. 13 kD, heparan sulfates and its derivatives, oligo- and polysaccharides of the erythrocyte glycol calyx, oligosaccharides, polysaccharides, completely desulfated and N-reacetylated heparin, desulfated and N-reacetylated heparin, N-carboxymethylated and/or partially N-acetylated chitosan, polyacrylic acid, polyether ether ketones, polyvinyl pyrrolidone and/or polyethylene glycol as well as mixtures of these compounds.

The inventive methods are suitable for the coating of for example endoprosthesis, and particularly stents such as coronary stents, vascular stents, trachea stents, bronchial stents, urethra stents, esophageal stents, bile duct stents, kidney stents, small intestine stents, colon stents, cerebral stent, pharynx stent, periphery stent and other stents. Moreover, spirals, catheters, cannulas, tubes, guide wires, as well as generally tubular or hose-like implants or parts of the aforementioned medical products can be coated according to the invention.

The endoprosthesis and particularly the stent may consist of current materials such as medical stainless steel, titanium, chrome, vanadium, tungsten, molybdenum, gold, iron, cobalt-chrome, Nitinol, magnesium, iron, alloys of the aforementioned metals as well as of bioresorbable metals and metal alloys such as magnesium, zinc, calcium, iron and so on as well as of polymeric material and preferably resorbable polymeric material such as chitosan, heparans, polyhydroxy butyrates (PHB), polyglycerides, polylactides and co-polymers of the afore-mentioned compounds. A catheter can be manufactured of any of the current materials in particular polymers such as polyamide, polyether, polyurethane, polyacrylates, polyethers and other polymers.

The coated medical products are used especially for keeping open all tubular structures, such as the urinary tract, oesophagus, trachea, bile duct, kidney ducts, blood vessels in the entire body including the brain, nose, duodenum, pylorus, small and large intestine, but also for keeping open artificial outlets such as used for the intestines or the trachea and also for keeping open long-term necessary artificial in- and outlets.

Thus the coated medical products are suitable for preventing, reducing or treating stenoses, restenoses, in-stent restenoses, arteriosclerosis, atherosclerosis, tumors, fistula formation, formation of cysts, aneurysm, bleeding in surrounding tissue and all other forms of vascular occlusions, vascular constrictions, vascular dilations and injuries of passages or outlets or artificial in- and outlets.

A further embodiment of the present invention relates to an endoprosthesis with a porous wall of synthetic polymer, wherein microparticles are embedded in the wall of the prosthesis on the surface of which blood coagulation inhibitors are immobilized. The blood coagulation inhibitors are preferably immobilized on the surface of the microparticles via so-called linkers (spacer molecules). Generally, the linkers are not covalently, but preferably adsorptively bound to the microparticle. The blood coagulation inhibitors are preferably covalently bound to the linkers. The covalent bondage is normally based on a chemical condensation reaction between functional groups of the linkers and suitable reactive groups of the inhibitors, for example hydroxy and/or amino groups. Through bondage with the linkers the blood coagulation inhibitors are at a certain distance to the microparticles. Thereby activity impairments of the inhibitors can be widely avoided. The immobilization of the linker-inhibitor conjugate on the microparticle surfaces is preferably based on adsorptive, particularly electrostatic interactions between the linkers and the microparticle surfaces.

In other preferred embodiments the linkers are polymeric molecules, conveniently with a linear structure. Preferably, these linkers are oligo- or polyalkylene glycols, in particular polyethylene glycol (PEG). The blood coagulation inhibitors are preferably serine protease inhibitors, in particular thrombin inhibitors. Thrombin is the key enzyme of plasmatic blood coagulation, cleaving fibrinogen to monomeric fibrin. The latter is polymerizing in the following and cross-links blood components adhered at the vascular wall inside to a thrombus.

### DESCRIPTION OF FIGURES

FIG. 1 shows a PLGA thread-tangle around a partially pre-expanded stent, having been crimped and expanded after coating with the thread-tangle. It can be easily recognized that the PLGA sleeve has stayed intact.

FIG. 2 shows a thread-tangle coated stent with micropores (d=200 μm; d denotes the average pore diameter).

FIG. 3 shows, in comparison to FIGS. 1 and 2, a not pre-expanded endoprosthesis with a burst open PLGA thread-tangle coating after crimping and expansion attempts. The stent was overextended such that the thread-tangle coating ruptured, allowing a good look at the thread-like coating structure. Under physiological conditions such a stent overextension does not occur so there is no danger that the thread-tangle coating ruptures.

FIG. 4 shows a tumor cell that due to its size is not able to penetrate to the other side of the thread-tangle coating.

FIG. 5 shows a REM-picture of a PU-fiber-web or rather fiber-tangle manufactured by spraying method on stainless steel gauze (1000× magnification). The white circles correspond to approximately 5 μm and shall give an impression of the fiber diameter. The flat areas are formed by agglutination of overlying fibers during the spraying process. The estimated pore size of the smallest pores is between 2 and 5 μm for both materials (Estimation in 10 k-pictures according to the small circles corresponding to approximately 5 μm). The structure of the inner and outer surface of the material does not differ substantially.

FIG. 6 shows a REM-picture of a PU-fiber-web or rather fiber-tangle manufactured by spraying method on stainless steel gauze (800× magnification). The flat areas are formed by agglutination of overlying fibers during the spraying process. The estimated pore size of the smallest pores is between 2 and 5 μm for both materials (Estimation in 10 k-pictures according to the small circles corresponding to approximately 5 μm). The structure of the inner and outer surface of the material does not differ substantially.

FIG. 7 shows the endoprosthesis in different phases of coating. A) Endoprosthesis before coating, mounted horizontal on a rod of the coating device; B) coated endoprosthesis, mounted horizontal on a rod of the coating device; C) coated endoprosthesis.

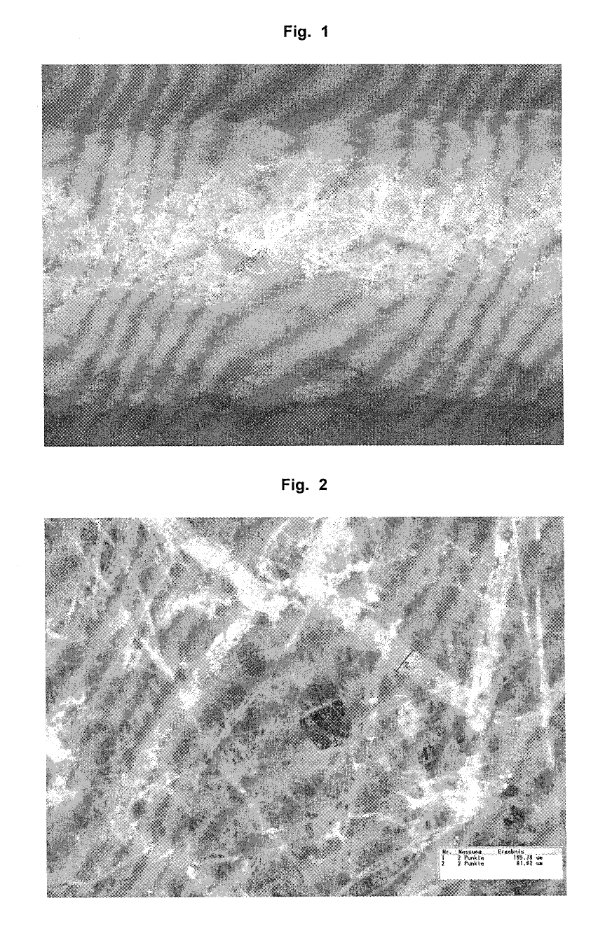

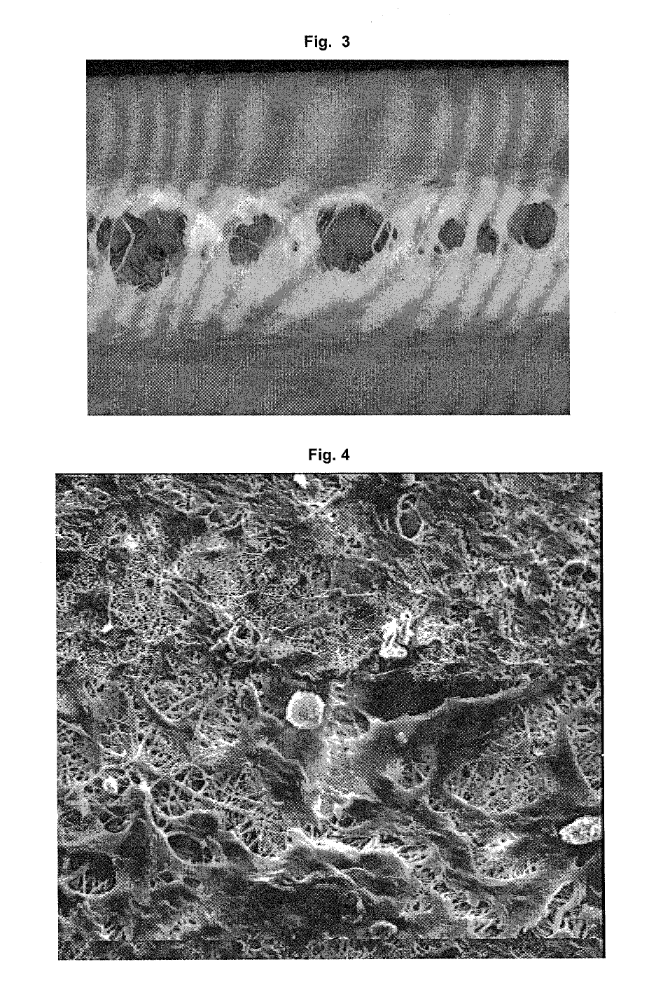

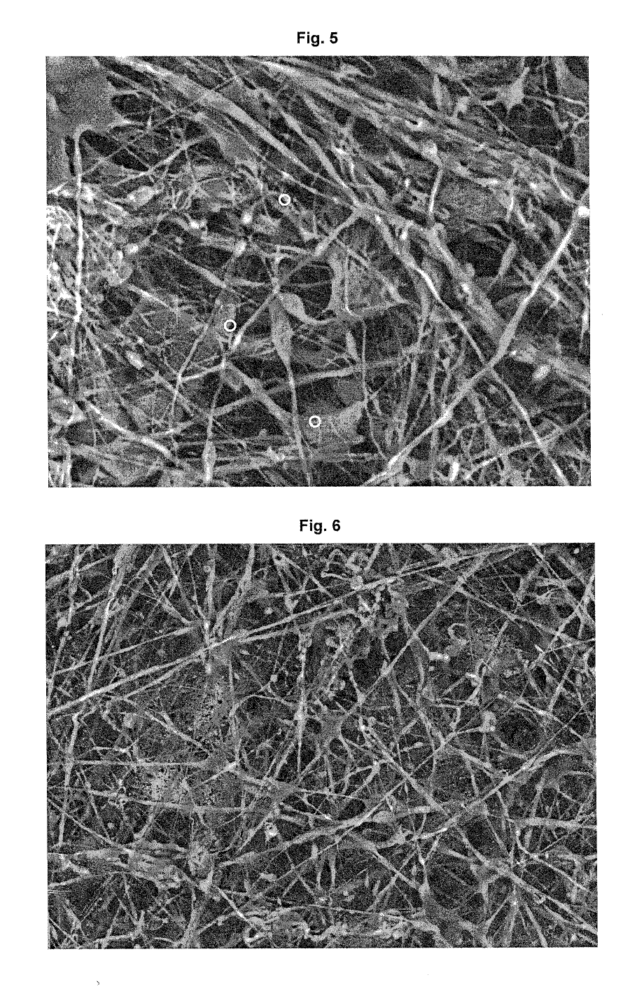

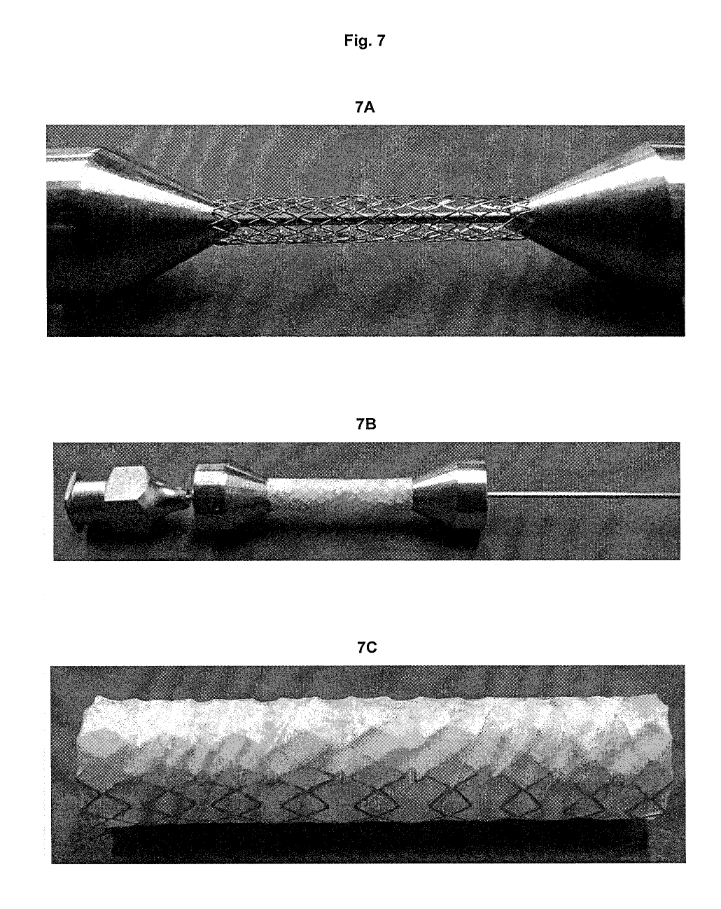

### EXAMPLES

### Example 1

### Pre-Coating of the Struts of the Endoprosthesis with a Polymer

The struts of an endoprosthesis were spray-coated with a 0.5% PLGA solution. To this aim, the stent is hung horizontally on a thin metal rod which is stuck on the rotational axis of the rotation and forward feed device, rotating with a defined rotatory speed. At a defined amplitude of the forward feed and rotatory speed and a defined distance between stent and nozzle the stent is sprayed with the spray solution. After drying at room temperature and storing in the exhaust hood over night it is weighed again. The pre-coating of the stent struts or endoprosthesis struts provides for a better adhesion of the thread-tangle on the struts.

### Example 2

### Full-Size Pre-Coating of the Struts of the Endoprosthesis with an Anti-Proliferative Active Agent Containing Polymer

Spray solution: 145.2 mg PLGA or polysulfone and 48.4 mg rapamycin or a 33% spray solution of a corresponding active agent combination of rapamycin (amount 20%-90%) with one or more further active agents such as paclitaxel, cyclosporine A, thalidomide, fusadil etc. are filled up with chloroform to 22 g.

This spray solution is applied on the stent as already described in example 1.

The stent can be a bare stent, a hemocompatible coated stent and/or a stent coated with an active agent layer by spray or dipping method.

The spray solution for coating merely the struts has in general another active agent than the following thread-tangle spray coating.

### Example 3

### Pre-Coating of the Endoprosthesis on the Example of a Transurethral or Suprapubic Catheter with an Anti-Bacterial Active Agent Containing Polymer

Solution: 144.5 mg PVP and a 32% spray solution of a corresponding anti-bacterial and anti-fungicide active agent combination (e.g. erythromycin and terbinafin 3:1 w:w) is filled up with chloroform to 22 g.

This spray solution is applied to the surface as described in example 1 full-size, uniformly and without any gaps according to the spray method (dipping method also possible).

### Example 4

### Full-Size or Strut-Interstices-Overlapping Full-Size Coating of the Endoprosthesis with a PLGA Thread-Tangle

After drying the partially pre-expanded endoprosthesis is sprayed with a PLGA solution containing 3% chloroform on the same spray coating device as in Example 1 in order to apply a dense moisture permeable thread-tangle.

### Example 5

### Production of a Full-Size or Strut-Interstices-Overlapping Full-Size Thread-Tangle Coated Endoprosthesis with a Smooth Interior Wall and PU-Thread-Tangle Coating on the Exterior Surface

An endoprosthesis is firmly mounted on a polished stainless steel rod and dipped into a viscous polyurethane (PU) solution in THF (ca. 16%) (e.g. chronoflex C 65D from Avansource Biomaterials Inc.).

On the slightly dried surface an uniform thread-tangle layer is applied in the following with a 6% PU solution in THF by means of the spraying device (e.g. Chronoflex C 80A). After drying the such thread-tangle coated stent is removed carefully from the metal rod.

### Example 6A

### Thread-Tangle Coating on an Endoprosthesis Crimped on a Balloon Catheter

The pre-treated stent is crimped on a balloon catheter and subsequently full-size coated with a 5% PLGA spraying solution (Resomer RG504H from Evonik with an inherent viscosity of 0.54 dl/g) in chloroform according to example 2.

### Example 7A

### Strut-Interstices-Overlapping Full-Size Coating of Stents with a PDLG-Thread-Tangle

Each 10 stents were pre-sprayed on the struts only with a 0.5% PDLG-solution (Purasorb PDLG 5010 from PURAC with an inherent viscosity of 1.03 dl/g) this pre-coating ensuring a better adhesion of the thread-tangle on the struts. After drying the stents were sprayed with a 3% PDLG-solution to apply a dense thread-tangle. The coating was sprayed over the right and left edges of the stent such that the turning points lay outside of the stent.

The PLGA-thread-tangle coating on the non-expanded stent as well as the coating of the 100% pre-expanded stent ruptured after crimping on the balloon catheter and expansion to 4 mm diameter. The PDGL-thread-tangle coating of the 50% pre-expanded stent remained intact during crimping and expansion. The functionality of the coating of the 50% pre-expanded stent was still unchanged after storage for 5-days without an inert atmosphere.

### Example 6B

### Hemocompatible Coating of an Endoprosthesis with Desulfated Reacetylated Heparin

Non-expanded stents made of medical stainless steel LVM 316 are degreased with acetone and ethanol in the ultrasound bath for 15 minutes and dried in the drying cabinet at 100° C. Subsequently, they are dipped into a 2% 3-aminopropyl triethoxysilane solution in an ethanol/water mixture (50/50 (v/v)) for 5 minutes and then dried at 100° C. for 5 minutes. Afterwards the stents are washed overnight in demineralized water.

3 mg desulfated and reacetylated heparin are solved at 4° C. in 30 ml 0.1 M MES buffer (2-(N-morpholino)ethanesulfonic acid) pH 4.75 and 30 mg N-cyclohexyl-N′-(2-morpholinoethyl)carbodiimide-methyl-p-toluene sulfonate are added. The stents are stirred in this solution at 4° C. for 15 hours. Afterwards it is rinsed with water, 4 M NaCl solution and water for 2 hours each.

### Example 7B

### Hemocompatible Coating of an Endoprosthesis Coated with a Thread-Tangle of Polyurethane

The same method for hemocompatible coating of surfaces as shown in example 6B and 3 can be applied on the thread-tangle of e.g. PU and thereby produce an endoprosthesis with a hemocompatible surface with a thread-tangle.

### Example 8

### Manufacturing of an Endoprosthesis with a Smooth Interior Wall and Sprayed Exterior Wall on the Example of Polyurethane

A polished stainless steel rod is used as carrier material for the dipping/spraying process for manufacturing the vascular prosthesis of polyurethane.

The metal rod is initially dipped in a viscous PU-solution (e.g. carbothane PC-3575A) in THF in order to obtain a smooth interior wall. Subsequently, a 6% polyurethane-THF-solution is sprayed on the pre-coated metal rod. After drying the endoprosthesis is incubated for 30 min in a bath with SDS-solution at 60° C. and then is detached from the metal rod. The so obtained endoprosthesis has a wall strength of 1 mm.

The wall strength is adjustable through the spraying process. The desired range of the wall strength is preferably between 1 and 1.5 mm. The diameter as well as the length of the endoprosthesis is variable and depends from the diameter and length of the stainless steel rod.

### Example 9

### Coating of Endoprosthesis with a Thread-Tangle of Polycarbonaturethane with Admixture of a Tenside (Tween 20)

For the spray-thread-tangle 1.5% to 6% polycarbonat urethane solutions in THF with an amount of tenside of 5%, 10% and 20% based on the proportions of solids in the solutions is manufactured.

During the coating with polycarbonat urethane-tenside-THF-solution the cylinder is moved back and forth in a longitudinal direction with a defined speed and at the same time is rotated around its longitudinal axis.

The higher the polymer concentration in spraying solution the thicker are the resulting threads. At low concentrations only very thin threads develop, wherein the structure is agglutinated by spray solutions droplets.

With increasing layer thickness the thread-tangles display a better wetting and spreading behavior for water. (However, the different concentrations of the tenside scarcely have any influence on the spreading behavior of water or water-like liquids or the wetting behavior of the thread-tangle surface.)

The thread-tangle is applied as uniformly as possible. Depending on the sprayed endoprosthesis the layer thickness is varied. In case of the herein described surfaces it is for example not thicker than 20 μm.

### Example 10

### Coating of an Expandable Esophagus Stent with a Molecular Permeable Thread-Tangle of Biostable Polymeric Fibers

Spraying solution with a high amount of a hydrophilic polymer:

Polyethersulfone/PVP—solution: 24.0 mg PS and 1.4 mg PVP are weighed and filled up with chloroform to 3 g→0.80% PS, 0.047% PVP

Optionally, according to example 1 only a strut coating basic layer of polyethersulfone may be applied with or without active agent, with or without hydrophilic polymeric additive to the polyethersulfone.

Spraying Solution with Active Agent Examples
a) PS/simvastatin/PVP-solution:
   23.2 mg PS, 8.8 mg simvastatin and 3.2 mg PVP are weighed and filled up to 4 g with chloroform→0.58% PS, 0.22% simvastatin, 0.08% PCP
   b. 13.2 mg PS and 4.4 mg paclitaxel are weighed and filled up to 2 g with chloroform→0.66% PS, 0.22% paclitaxel
   c. 11.6 mg PS, 3 mg PVP and 4.4 mg paclitaxel are weighed and filled up to 2 g with chloroform→0.58% PS, 0.15 PVP, 0.22% paclitaxel

Active agents or active agent combinations can be solved in chloroform up to ca. 40 percent by weight with polyethersulfone and the admixture of an intrafilamentous permeability enhancing hydrogel such as PVP, PVA and other hydrophilic polymers, resulting in a solution with at least 0.04% hydrogel that can be applied to an endoprosthesis.

The pores formed by the thread-tangle are loaded afterwards with rapamycin by dipping the stent coated with the thread-tangle in an active agent solution (2% solution in a volatile solvent).

### Example 11

### Interfilamentary Active Agent Containing Thread-Tangle Coating of an Endoprosthesis

The endoprosthesis according to example 8, but without the addition of a tenside, is coated with the thread-tangle. Subsequently, the filament interstices are filled with an active agent containing solution by dipping method and exploiting the capillary properties of the coating.

b) Likewise it is possible to apply a pure active agent layer on the thread-tangle coating by spraying the surface with a solution with a defined amount of active agent and subsequent drying.

c) The thread-tangle coating can also be loaded in a most easily manner with another or the same active agent by dipping it in an active agent containing solution. By means of capillary forces the pores of the thread-tangle are filled with active agent.

d) In the same way the different active agents can be applied separately, for example e) filling of the pores of the thread-tangle with agents, will accelerate the uptake of active agent in the vascular wall.

e) Filling of the pores with short-term biodegradable polymers such as PLGA 50/50, that can release the active agent controlled and time-displaced.

f) Combination of the aforementioned possible variations.

1. Endoprosthesis with a surface having at least partially a coating of a polymeric close-meshed or tight-meshed thread-tangle.

2. Endoprosthesis according to claim 1, wherein the thread-tangle consists of at least one biostable or biodegradable polymer selected from the group comprising or consisting of:
Polyurethane, polyethylene terephthalate, polyvinyl chloride, polyvinyl ester, polyvinyl acetales polyamides, polyimides, polyacrylnitriles, polyethers, polyesters, polyamino acids, polysaccharides, polylactides, polyglycolides, polylactide-co-glycolides, chitosans, carboxyalkyl chitosans, collagen, polyethylene glycol, polyvinyl pyrrolidone, polyphosphazenes, polystyrenes, polysulfones as well as derivatives, block polymers, co-polymers and mixtures of the aforementioned polymers.

3. Endoprosthesis according to claim 1, wherein the thread-tangle coating has meshes.

4. Endoprosthesis according to claim 3, wherein the meshes have an average transverse diameter in the range of 0.01 μm to 1.000 μm and/or an average longitudinal diameter in the range of 0.01 μm to 1.000 μm.

5. Endoprosthesis according to claim 1, wherein the threads of the thread-tangle coating are porous.

6. Endoprosthesis according to claim 1, wherein the thread-tangle coating has a porosity defined as air permeability of 1 to 150 ml air per square centimeter per minute at a pressure difference of 1.2 kPa.

7. Endoprosthesis according to claim 1, wherein the thread-tangle coating has a porosity defined as water permeability of 100 to 300 ml/(cm²*min) and in particular of 150 to 250 ml/(cm²*min).

8. Endoprosthesis according to claim 1, further comprising at least one antiproliferative, anti-inflammatory, antimigratory, antiphlogistic, anti-angiogenic, cytostatic, cytotoxic, anti-restenotic, anti-neoplasic, anti-bacterial and/or anti-mycotic agent.

9. Endoprosthesis according to claim 8, wherein the at least one antiproliferative, anti-inflammatory, antimigratory, antiphlogistic, anti-angiogenic, cytostatic, cytotoxic, anti-restenotic, anti-neoplasic, anti-bacterial and/or anti-mycotic agent is selected from the group comprising or consisting of:
Abciximab, acemetacin, acetylvismione B, aclarubicin, ademetionine, adriamycin, aescin, afromoson, akagerine, aldesleukin, amidorone, aminoglutethemide, amsacrine, anakinra, anastrozole, anemonin, aminopterine, antimycotics, antithrombotics, apocymarin, argatroban, aristolactam-All, aristolochic acid, ascomycin, asparaginase, aspirin, atorvastatin, auranofin, azathioprine, azithromycin, baccatine, bafilomycin, basiliximab, bendamustine, benzocaine, berberine, betulin, betulinic acid, bilobol, bisparthenolidine, bleomycin, bombrestatin, Boswellic acids and derivatives thereof, bruceanoles A, B and C, bryophyllin A, busulfan, antithrombin, bivalirudin, cadherins, camptothecin, capecitabine, o-carbamoyl-phenoxy-acetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type Natriuretic Peptide (CNP), cudraisoflavone A, curcumin, cyclophosphamide, cyclosporine A, cytarabine, dacarbazine, daclizumab, dactinomycin, dapson, daunorubicin, diclofenac, 1,11-dimethoxycanthin-6-one, docetaxel, doxorubicin, daunomycin, epirubicin, epothilones A and B, erythromycin, estramustine, etoposide, everolimus, filgrastim, fluoroblastin, fluvastatin, fludarabine, fludarabine-5′-dihydrogenphosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mitoxantrone, mycophenolate mofetil, β-lapachone, podophyllotoxin, podophyllic acid 2-ethylhydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies which inhibit muscle cell proliferation, bFGF antagonists, probucol, prostaglandins, 1-hydroxy-11-methoxycanthin-6-one, scopoletin, NO donors, pentaerythritol tetranitrate and sydnonimines, S-nitrosoderivatives, tamoxifen, staurosporine, □-estradiol, □-estradiol, estriol, estrone, ethinylestradiol, medroxyprogesterone, estradiol cypionates, estradiol benzoates, tranilast, kamebakaurin, terpenoides, verapamil, tyrosine kinase inhibitors, tyrphostins, paclitaxel and its derivatives, 6-α-hydroxy-paclitaxel, taxotere, mofebutazone, lonazolac, lidocaine, ketoprofen, mefenamic acid, piroxicam, meloxicam, penicillamine, hydroxychloroquine, sodium aurothiomalate, oxaceprol, β-sitosterin, myrtecaine, polidocanol, nonivamide, levomenthol, ellipticine, colcemid, cytochalasin A-E, indanocine, nocadazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1, antibiotics, cefadroxil, cefazolin, cefaclor, cefoxitin, tobramycin, gentamycin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyridamol, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, etoposide, dicloxacillin, tetracycline, triamcinolone, mutamycin, procainimide, retinoic acid, quinidine, disopyramide, flecamide, propafenone, sotolol, natural and synthetically obtained steroids, inotodiol, maquiroside A, ghalakinoside, mansonine, strebloside, hydrocortisone, betamethasone, dexamethasone, non-steroidal substances (NSAIDS), fenoprofen, ibuprofen, indomethacin, naproxen, phenylbutazone, antiviral agents, acyclovir, ganciclovir, zidovudin, clotrimazole, flucytosine, griseofulvin, ketoconazole, miconazole, nystatin, terbinafine, antiprotozoal agents, chloroquine, mefloquine, quinine, natural terpenoids, hippocaesculin, Barringtogenol-C21-angelat, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, Coronarin A, B, C und D, ursolic acid, hyptatic acid A, iso-iridogermanal, maytenfoliol, effusantin A, excisanin A and B, longikaurin B, sculponeatin C, kamebaunin, leukamenin A and B, 13,18-Dehydro-6-alpha-senecioyloxychaparrin, taxamairin A and B, regenilol, triptolide, cymarin, hydroxyanopterin, protoanemonin, cheliburin chloride, sinococuline A and B, dihydronitidine, nitidine chloride, 12-beta-hydroxypregnadien-3,20-dion, helenalin, indicine, indicine-N-oxide, lasiocarpine, inotodiol, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, bisparthenolidine, oxoushinsunine, periplocoside A, ursolic acid, deoxypsorospermin, psycorubin, ricin A, sanguinarine, manwu wheat acid, methylsorbifolin, sphatheliachromen, stizophyllin, mansonine, strebloside, dihydrousambaraensine, hydroxyusambarine, strychnopentamine, strychnophylline, usambarine, usambarensine, liriodenine, oxoushinsunine, daphnoretin, lariciresinol, methoxylariciresinol, syringaresinol, sirolimus and its derivatives such as biolimus A9, everolimus, myolimus, novolimus, pimecrolimus, ridaforolimus, tacrolimus FK 506, temsirolimus and zotarolimus, somatostatin, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, zeorin, vismione A and vismione B.

10. Endoprosthesis according to claim 1, wherein the endoprosthesis is provided with an exterior hemocompatible layer and/or an interior hemocompatible layer.

11. Endoprosthesis according to claim 1, wherein the endoprosthesis is a stent.

12. Endoprosthesis according to claim 1 for preventing, reducing or treating lesions of the wall of body passages, stenosis, restenosis, in-stent restenosis, late stent thrombosis, arteriosclerosis, vascular occlusions, vascular constrictions, constricted heart valves, aneurysms, artificial outlets and inlets to the human body and laying a lumen in the human body.

13. Method for coating of an endoprosthesis comprising the following steps:
a) providing an endoprosthesis,
b) solving a polymer in a volatile solvent,
c) applying of a thread-tangle of the polymer by means of spraying or electrospinning on the surface of the endoprosthesis.

14. Endoprosthesis according to claim 2, wherein the thread-tangle coating has meshes.

15. Endoprosthesis according to claim 2, wherein the threads of the thread-tangle coating are porous.

16. Endoprosthesis according to claim 2, wherein the thread-tangle coating has a porosity defined as air permeability of 1 to 150 ml air per square centimeter per minute at a pressure difference of 1.2 kPa.

17. Endoprosthesis according to claim 2, wherein the thread-tangle coating has a porosity defined as water permeability of 100 to 300 ml/(cm²*min) and in particular of 150 to 250 ml/(cm²*min).

18. Endoprosthesis according to claim 2, wherein the endoprosthesis is provided with an exterior hemocompatible layer and/or an interior hemocompatible layer.

19. Endoprosthesis according to claim 2, wherein the endoprosthesis is a stent.

20. Endoprosthesis according any to claim 2 for preventing, reducing or treating lesions of the wall of body passages, stenosis, restenosis, in-stent restenosis, late stent thrombosis, arteriosclerosis, vascular occlusions, vascular constrictions, constricted heart valves, aneurysms, artificial outlets and inlets to the human body and laying a lumen in the human body.
